(19) United States
(12) Patent Application Publication
(10) Pub. No.: US 2011/0046684 A1
(43) Pub. Date: Feb. 24, 2011

(54) Screw Assembly and Method

(75) Inventors: Mahmoud F. Abdelgany, Rockaway, NJ (US); Young Hoon Oh, Montville, NJ (US); Aaron Markworth, New York, NY (US)

Correspondence Address: Rahman LLC, 10025 Governor Warfield Parkway, Suite 110, Columbia, MD, 21044, US

(73) Assignee: Custom Spine, Inc., Parsippany, NJ (US)

(21) Appl. No.: 12/917,328

(22) Filed: Nov. 1, 2010

Related U.S. Application Data
(63) Continuation-in-part of application No. 11/045,908, filed on Jan. 28, 2005, now Pat. No. 7,862,594.
(60) Provisional application No. 60/548,543, filed on Feb. 27, 2004.
(60) Provisional application No. 60/565,658, filed on Apr. 27, 2004.

(51) Int. Cl. A61B 17/86

(52) U.S. Cl. 606/305

(57) ABSTRACT

A screw assembly and method includes a coupling member comprising a semi-bulbous end; a fixator component that receives the semi-bulbous end of the coupling member; a resisting member mounted in the coupling member and comprising a mating member; a connection pin comprising a resisting member socket operatively connected to the mating member of the resisting member; and a blocker that engages the coupling member.

### CROSS REFERENCE TO RELATED APPLICATION

This application is a continuation-in-part of U.S. patent application Ser. No. 11/045,908, filed on Jan. 28, 2005, which claims the benefit of U.S. Provisional Patent Application No. 60/548,543 filed on Feb. 27, 2004 and U.S. Provisional Patent Application No. 60/565,658 filed on Apr. 27, 2004, the contents of which in their entireties are herein incorporated by reference.

### BACKGROUND

1. Technical Field

The embodiments herein generally relate to medical devices and assemblies, and more particularly to an orthopedic surgical implant assembly used in the field of surgical lumbar, thoracic and cervical spine treatment.

2. Description of the Related Art

Surgical procedures treating spinal injuries are one of the most complex and challenging surgeries for both the patient and the surgeon. When there are various deformities, trauma, or fractures of the vertebra, surgeons may attempt to “fuse” them together by attaching screw-like devices into the pedicles of the spine and thereby connecting several vertebrae (typically two or more) using a semi-rigid rod. However, due to the complexity of the human anatomy, most surgeons must bend the rod (causing notches thereby reducing fatigue resistance) before placing them into two or more non-aligned pedicle screws in order to properly stabilize the pedicle screw assembly within the patient's body.

Depending on the purpose of the spine surgery, indications, and patient size, surgeons must pre-operatively choose between different spinal systems with differing rod sizes sometimes causing delays in surgery while waiting for more adequate systems to be sterilized. Some surgeons prefer monoaxial screws for rigidity, while some sacrifice rigidity for surgical flexibility in screw placement. Therefore, a system is needed to accommodate both theories. For example, during scoliosis surgery conventional polyaxial systems typically cannot lock into a desired position to persuade the spinal column into desired correction before final construct assembly.

Most conventional top loading polyaxial spine screws do not do enough to address cantilever failure of the assembly components. Additionally, most polyaxial screws generally do not offer enough flexibility because the rod sits too closely on top of the center of rotation. Furthermore, most top loading screw systems generally do not accommodate different rod sizes. Thus, there remains a need for a new and improved pedicle screw assembly capable of overcoming the limitations of the conventional designs thereby providing the surgeon with improved intra-operative flexibility and the patient with an improved prognosis for better and complete rehabilitation.

### SUMMARY

In view of the foregoing, an embodiment herein provides an assembly comprising a coupling member comprising a semi-bulbous end; a fixator component that receives the semi-bulbous end of the coupling member; a resisting member mounted in the coupling member, the resisting member comprising a mating member; a connection pin comprising a resisting member socket operatively connected to the mating member of the resisting member; and a blocker that engages the coupling member.

The coupling member may comprise a slot that receives a longitudinal member; and a bottom portion operatively connected to the semi-bulbous end. The fixator component may comprise a concave socket that receives the semi-bulbous end of the coupling member. The fixator component may comprise an anchor end opposite the concave socket, and the anchor end attaches to a bone. The connection pin engages the fixator component and a bottom portion of the longitudinal member. The blocker secures a top portion of the longitudinal member. The semi-bulbous end may comprise a coupling member flat end, and the semi-bulbous end sits flush with the resisting member. The connection pin may comprise a connection pin flat end, and the connection pin flat end may sit flush with the resisting member. The resisting member may comprise a mechanically harder material than the connection pin.

The coupling member and the fixator component may comprise a first material, and the resisting member may comprise a material having a higher material hardness and compressive yield strength than the first material. The resisting member may comprise a plurality of separately configured parts matingly attached together. The coupling member may comprise a plurality of opposed upright ends separated by the slot. Each of the opposed upright ends may comprise an inner wall and an outer wall, and any of the inner wall and the out wall may comprise wall threads, and the outer wall may comprise grooves. The blocker may comprise blocker threads dimensioned and configured to mate with the wall threads. The semi-bulbous end of the coupling member may comprise a plurality of slots terminating at an opening at a tip of the semi-bulbous end. The semi-bulbous end of the coupling member may comprise a gap that receives the connection pin.

Another embodiment provides a pedicle fixation assembly comprising a screw head comprising a polyaxial rotatable connecting end comprising a curved outer surface that forms a semi-bulbous body, wherein the semi-bulbous body comprises a substantially first flat surface; a resisting member comprising a substantially second flat surface sitting flush with the first flat surface; and a convexed surface curved to match a contour of the curved outer surface of the polyaxial rotatable connecting end of the screw head. A bone fixator component comprises a female concave semi-spherical socket for receiving the screw head. A pin is provided for engaging the screw head, the resisting member, and the bone fixator component; and a blocker is provided for engaging the screw head and for securing the longitudinal member. The resisting member may comprise a resisting cap; and a resisting anchor comprising a socket for receiving the resisting cap.

Another embodiment provides a method of assembling a pedicle fixation assembly, the method comprising attaching a coupling member to a bone fixator component; securing the bone fixator component to a bone; securing a resisting member in the coupling member; securing a connection pin to the coupling member; engaging the connection pin and the resisting member with the fixator component; inserting a longitudinal member in the coupling member; and connecting a blocker to the coupling member. The coupling member may comprise a male semi-spherical end and the fixator component comprises a female concave semi-spherical socket for receiving the coupling member.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates an exploded view of a screw assembly according to a first embodiment herein;

FIG. 2 illustrates a perspective view of a fully assembled screw assembly according to a first embodiment herein;

FIG. 3 illustrates a cross-sectional view of a fully assembled screw assembly according to a first embodiment herein;

FIG. 4(A) illustrates a perspective view of a coupling member according to an embodiment herein;

FIG. 4(B) illustrates a top view of the coupling member of FIG. 4(A) according to an embodiment herein;

FIG. 4(C) illustrates a side view of the coupling member of FIG. 4(A) according to an embodiment herein;

FIG. 4(D) illustrates a cross-sectional view cut along line A-A of the coupling member of FIG. 4(C) according to an embodiment herein;

FIG. 5(A) illustrates a cross-sectional view of a saddle connection pin according to a first embodiment herein;

FIG. 5(B) illustrates a side view of a saddle connection pin according to a first embodiment herein;

FIG. 5(C) illustrates a top view of a saddle connection pin according to a first embodiment herein;

FIG. 5(D) illustrates a perspective view of a saddle connection pin according to a first embodiment herein;

FIG. 6(A) illustrates a cross-sectional view of a resisting member according to an embodiment herein;

FIG. 6(B) illustrates a side view of a resisting member according to an embodiment herein;

FIG. 6(C) illustrates a top view of a resisting member according to an embodiment herein;

FIG. 6(D) illustrates a perspective view of a resisting member according to an embodiment herein;

FIG. 7(A) illustrates a side view of a securing member according to an embodiment herein;

FIG. 7(B) illustrates a cross-sectional view of a securing member according to an embodiment herein;

FIG. 7(C) illustrates a top view of a securing member according to an embodiment herein;

FIG. 7(D) illustrates a perspective view of a securing member according to an embodiment herein;

FIG. 8(A) illustrates a top view of a longitudinal member according to an embodiment herein;

FIG. 8(B) illustrates a side view of a longitudinal member according to an embodiment herein;

FIG. 8(C) illustrates a front view of a longitudinal member according to an embodiment herein;

FIG. 8(D) illustrates a perspective view of a longitudinal member according to an embodiment herein;

FIG. 9(A) illustrates a side view of a fixator component according to an embodiment herein;

FIG. 9(B) illustrates a cross-sectional view of a fixator component according to an embodiment herein;

FIG. 9(C) illustrates a top view of a fixator component according to an embodiment herein;

FIG. 9(D) illustrates a perspective view of a fixator component according to an embodiment herein;

FIG. 10(A) illustrates an exploded view of screw assembly according to a second embodiment herein;

FIG. 10(B) illustrates a cross-sectional view of the fully assembled screw assembly shown in FIG. 10(A) according to a second embodiment herein;

FIG. 11(A) illustrates a cross-sectional view of a resisting member assembly according to a second embodiment herein;

FIG. 11(B) illustrates a perspective view of a resisting anchor of the resisting member assembly of FIG. 11(A) according to a second embodiment herein;

FIG. 11(C) illustrates a perspective view of a resisting cap of the resisting member assembly of FIG. 11(A) according to a second embodiment herein; and

FIG. 12 is a flow diagram illustrating a preferred method according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

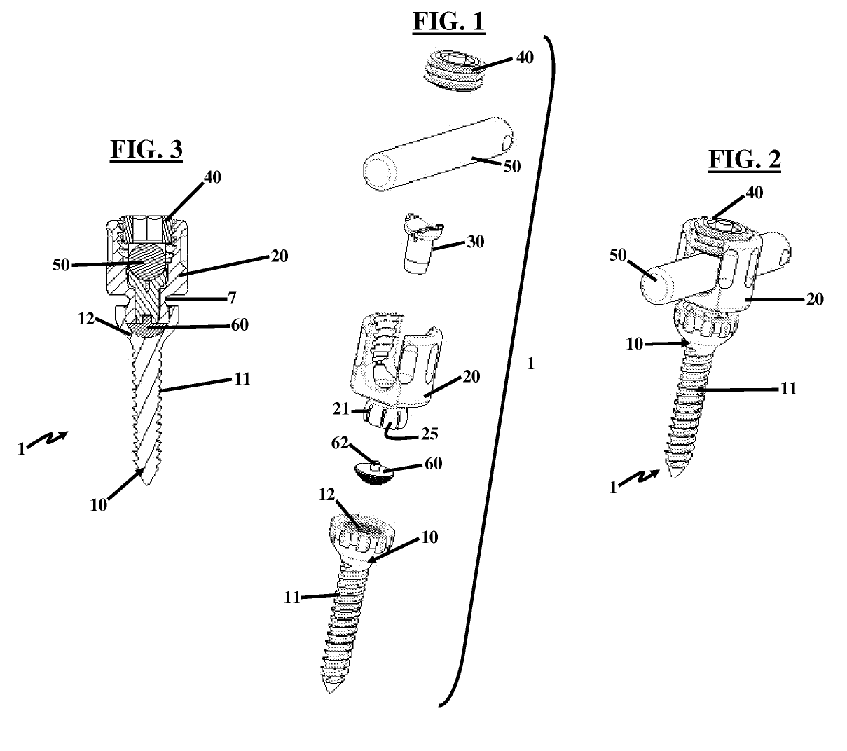

As mentioned, there remains a need for a new and improved pedicle screw assembly capable of overcoming the limitations of the conventional designs thereby providing the surgeon with improved intra-operative flexibility and the patient with an improved prognosis for better and complete rehabilitation. The embodiments herein address this need by providing an improved polyaxial pedicle screw device and method of assembly capable of accommodating multiple longitudinal member diameters and withstanding higher failure strengths as well as providing additional locking resistance to the assembly. Referring now to the drawings and more particularly to FIGS. 1 through 12 where similar reference characters denote corresponding features consistently throughout the figures, there are shown preferred embodiments.

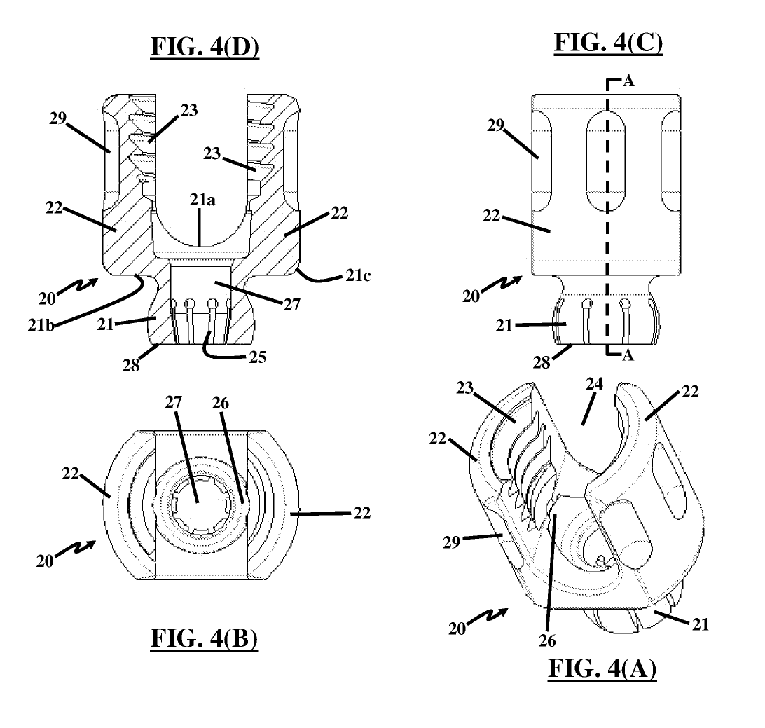

FIGS. 1 through 3, with reference to FIGS. 4(A) through 4(D), illustrate a pedicle screw assembly 1 according to a first embodiment herein. The screw assembly 1 comprises a fixator component (e.g., a bone screw) 10 having a threaded end 11 for engaging a bone (not shown) and a concave female socket 12 for engaging and receiving the coupling member 20. In another embodiment, the fixator component may be embodied as a hook mechanism (not shown).

As implemented, the coupling member 20 is first snapped into place in the fixator component 10 as shown in FIG. 2. Then, as shown in FIG. 3 the saddle connection pin 30 snaps into place in the lower base portion 25 of the coupling member 20, which includes a groove 26 (not shown in FIG. 1, but is best seen in FIGS. 4(A) through 4(D)) for receiving the saddle connection pin 30. In the manufacturing process, once the saddle connection pin 30 snaps into place, the screw assembly 1 is prepared for ultra sonic cleaning to remove any impurities and subsequently may be shipped in this manufactured format (with the saddle connection pin 30 connected to the coupling member 20, which is connected to the fixator component 10).

FIG. 3 also shows that the female spherical pocket 12 of the fixator component 10 has an undercut 7 to allow the coupling member 20 to pivot freely but not to disassemble once the expanding saddle connection pin 30 is inserted. The thread 11 of the fixator component 10 may be a multiple lead thread to allow faster insertion into a bone. This thread 11 may be tapered on the minor diameter while cylindrical on the major diameter to allow a new “bite” with every turn and to accommodate more thread depth towards the bottom of the fixator component 10 for the cancellous bone.

Once the fixator component 10 is inserted into the bone, a longitudinal member 50, which may be embodied as a rod, bar, etc. and securing member 40 are inserted into the screw assembly 1, as illustrated in FIGS. 2 and 3. In one embodiment herein, the coupling member 20 can accommodate 5.5 mm as well as 6.0 mm rods, which is advantageous over conventional screw assemblies that are limited to accepting only rods of a uniform dimension. FIG. 2 illustrates the assembled view of the screw assembly 1 in the straight monoaxial direction. In one embodiment, the threads 11 of the fixator component 10 are double lead, which provides greater surface contact with the bone, but drives at 4 mm/revolution. While not shown in FIG. 2, in one embodiment herein, the maximum angulation is 25 degrees/side, but the medial correction/travel of the longitudinal member 50 is 3.8 mm/side, which is nearly twice of what most conventional screws offer.

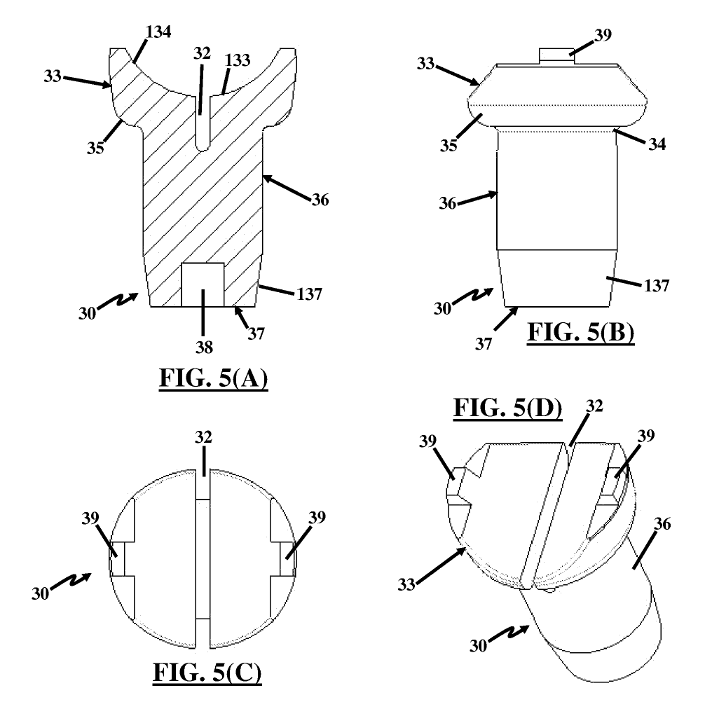

In addition, FIGS. 1 through 3 illustrate a schematic diagram of a locking process of the screw assembly 1 according to one embodiment herein. In the embodiment shown in FIGS. 1 through 3, the semi-spherical portion 21 (e.g., a polyaxial rotatable connecting end) of the coupling member 20 has slots 25 to allow the semi-spherical portion 21 to be snap-inserted into the female socket 12 of fixation component 10. In addition, the saddle connection pin 30 is placed on the coupling member 20 (e.g., in groove 26, as shown in FIGS. 4(A) through 4(D)) to prevent the coupling member 20 from disengaging the fixation component 10. As shown in FIG. 1, the one-piece resisting member 60 is placed inside the female socket 12 of the fixation component 10. In addition, the connecting portion 62 (shown in FIG. 5(A) of the resisting member 60 is inserted into the resisting member socket 38 (not shown in FIG. 1, but is best seen in FIGS. 4(A) through 4(D)) of the saddle connection pin 30. Consequently, saddle connection pin 30 is located between the longitudinal member 50 and the resisting member 60. In addition, the longitudinal member 50 connects and is located between neighboring screw assemblies (not shown).

FIGS. 4(A) through 4D, with reference to FIGS. 1 through 3, illustrate various views of coupling member 20. FIG. 4(A) illustrates a perspective view of the coupling member 20. FIG. 4(B) illustrates a top view of the coupling member 20. FIG. 4(D) is a cross-sectional view from cut-line “AA” of FIG. 4(C). As shown in FIGS. 4(A) through 4(D), the coupling member 20 includes a semi-bulbous (e.g., semi-spherical) male end 21 for engaging the concave female socket 12 (shown in FIG. 1) of fixator component 10 (shown in FIGS. 1 through 3). In the embodiment shown in FIGS. 4(C) through 4(D), semi-bulbous male end includes a generally flat lower surface 28. As shown in FIG. 1, resisting member 60 couples with coupling member 20. In one embodiment herein, flat surface 20 is flush with the generally flat surface 64 of resisting member 60 (not shown in FIGS. 4(A) through 4(D)). The coupling member 20 also includes a pair of upright ends 22 opposite the semi-bulbous male end 21 and connected by a bottom curved inner portion 21a, wherein the upright ends 22 comprise a threaded inner portion 23 for engaging the securing member 40 (shown in FIGS. 1 through 3). The coupling member 20 further includes a substantially flat bottom portion 21b that connects to the semi-bulbous male end 21. The upright ends 22 may connect to the bottom portion 21b by a curved elbow connection 21c. In another embodiment, not shown, the threaded portion 23 may be located on the outer side of the upright ends 22. Furthermore, the coupling member 20 includes a generally open U-shaped inner portion 24 for receiving the saddle connection pin 30 (shown in FIGS. 1 through 3) and the longitudinal member 50 (shown in FIGS. 1 through 3). The semi-bulbous male end 21 of the coupling member 20 includes a plurality (for example, four or more) slots 25 that allow the semi-bulbous male end 21 to outwardly expand into the female socket 12 (shown in FIG. 1) of the fixator component 10 (as shown in FIGS. 1 through 3) at any allowable angle once the saddle connection pin 30 (shown in FIG. 1) is forced through the hole 27 of the coupling member 20.

Since the coupling member 20 is pivoting inside the female socket end 12 of the fixator component 10, the screw assembly 1 is allowed to be inserted deeper into the bone without having the bone or anatomy prematurely limit the range of angulations of the coupling member 20. The coupling member 20 further includes external features or cuts 29 that assist in accommodating surgical instrumentation (not shown) during manipulation and assembly during the surgical procedure. These cuts 29 allow various instruments (not shown) to firmly and positively hold and manipulate the coupling member 20 on one side or both sides of coupling member 20.

One embodiment of the saddle connection pin 30 is shown in FIGS. 5(A) through 5(D), with reference to FIGS. 1 through 4(D). The saddle connection pin 30 provides a proper seat for the longitudinal member 50 (shown in FIGS. 1 through 3) and avoids notching a typical titanium longitudinal member 50 (titanium is very notch sensitive). Furthermore, the saddle connection pin 30 accommodates multiple sizes of longitudinal members 50 in the same screw assembly 1, which is unique compared to conventional titanium systems because of the above-mentioned notching factors. The saddle connection pin 30 is configured with a slot 32 through the center to allow lateral expansion of the upper portion (head) 33 of the saddle connection pin 30. The bottom 35 of the saddle connection pin head 33 is angled to fit into hole 27 of the coupling member 20. The saddle connection pin 30 initially expands the semi-bulbous male end 21 of the coupling member 20 into the female socket 12 in the fixator component 10 causing the screw assembly 1 to lock or start locking (i.e., causing the semi-bulbous male end 21 of the coupling member 20 to lock in the female socket 12 of the fixator component 10). The saddle connection pin 30 then “digs” into the female socket 12 of the fixator component 10 to provide a secondary locking force to avoid bending failure of the screw assembly 1.

As discussed above, upper portion 33 of saddle connection pin 30 includes a slot 32, which is configured from the lowest area 133 of the upper portion 33 into the upper area 134 of the upper portion 33 of the saddle connection pin 30. The lower portion 36 of the saddle connection pin 30 terminates with a generally flat end 37 and resisting member socket 38, which allows for resisting member 60 (shown in FIG. 1) to connect into the female socket 12 of the fixator component 10. In the embodiment shown in FIGS. 5(B) through 5(D), saddle connection pin 30 includes two generally flat upper opposed ends 39 to more matingly configure with the geometry of the coupling member 20 and the longitudinal member 50. The ends 39 fit into the grooves 26 of the coupling member 20.

In one embodiment, the saddle connection pin 30 may be configured such that the upper portion 33 comprises titanium and the lower portion 36 comprises a ceramic material. Accordingly, the ceramic material of the lower portion 36 of the saddle connection pin 30 has a higher hardness and compressive yield strength than the comparative hardness and compressive yield strength of Ti₆Al₄V, which is the material which may be used in constructing the coupling member 20 and fixator component 10.

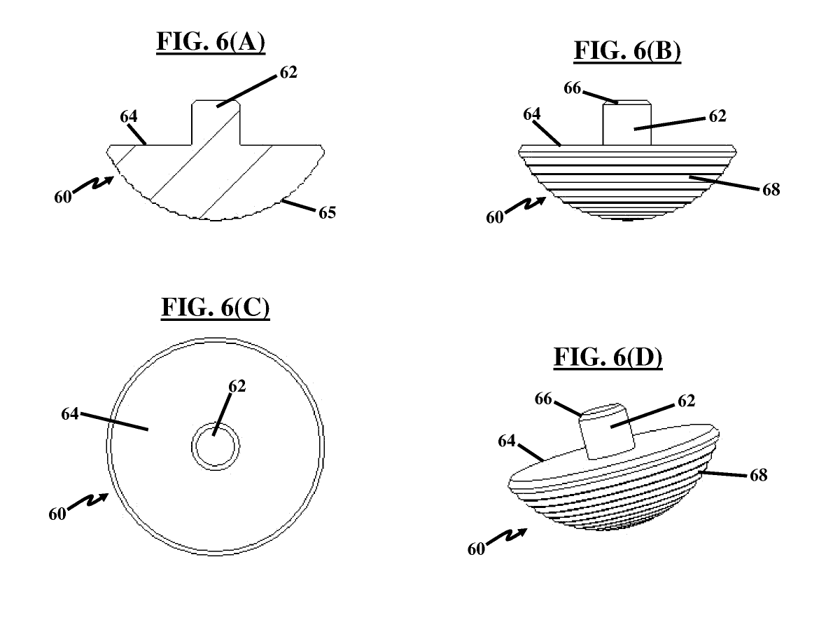

FIGS. 6(A) through 6(D), with reference to FIGS. 1 through 5(D), illustrate various views of resisting member 60 according to an embodiment herein. The resisting member 60 includes a mating member 62 protruding/extending outwardly from a generally flat surface 64 to securely and evenly couple to saddle connection pin 30 (shown in FIGS. 5(A) through 5(D)) via resisting member socket 38 and flat end 37. Resisting member 60 also includes a convexed surface 65 that in curved to create a full sphere when mated with the male semi-bulbous end 21 (shown in FIG. 1). Moreover, the convexed surface 65 is curved to match the inner female socket 12 of the fixator component 10. In addition, the embodiment of resisting member 60 includes a chamfered edge 66 on mating member 62. As stated above, resisting member is placed in the female socket 12 of fixator component 10. To better secure resisting member into female socket 12, one embodiment of resisting member 60 includes ridges 68, as shown in FIGS. 6(B) and 6(D). The material properties of the resisting member 60 are such that it prevents the deformation on the saddle connection pin 30 before the saddle connection pin 30 gives the proper bending and penetrating effects onto the coupling member 20/fixator component 10 assembly. Examples of the types of materials used for the resisting member 60 include Zyranox™ and HIP Vitox™, both of which are available from Morgan Advanced Ceramics, United Kingdom.

As shown in FIGS. 5(A) through 6(D), the upper portion 33 of the saddle connection pin 30 includes a slot 32 in the seat portion 133 and tapered angled sidewalls 134. Preferably, the saddle connection pin 30; i.e., the upper portion 33 and the lower portion 36 are assembled last in the overall process discussed above. Specifically, the coupling member 20 snaps into the fixator component 10. Then, the resisting member 60 slides into the coupling member 20, and finally the saddle connection pin 30 is press fitted into the coupling member 20 keeping everything in place and oriented in a relaxed state. In addition, the lower portion 36 of the saddle connection pin 30 terminates with a series of cascading walls 137 having sloped angles, terminating with the flat end 37 for attachment into resisting member 60.

According to FIGS. 1 through 6(D), the embodiments herein provide a screw head (e.g., coupling member 20) comprising a slot (e.g., open U-shaped inner portion 24); a substantially flat bottom portion 21b); and an outwardly protruding and expandable round hollow semi-bulbous end (e.g., semi-bulbous male end 21) extending from the outer bottom portion 21b. A resisting member 60 is mounted to the semi-bulbous end 21 of the screw head (e.g., coupling member 20), and comprises a mating member (e.g., connecting portion 62). A bone fixator component 10 comprises a concave socket (e.g., female socket 12) that cups the expandable semi-bulbous end 21 of the screw head (e.g., coupling member 20) and the resisting member 60. A locking pin (e.g., saddle connection pin 30) expands the expandable semi-bulbous end 21 of the screw head (e.g., coupling member 20), couples to the resisting member 60 (e.g., through resisting member socket 38) and engages the bone fixator component 10. A blocker (e.g., securing member 40) engages the screw head (e.g., coupling member 20).

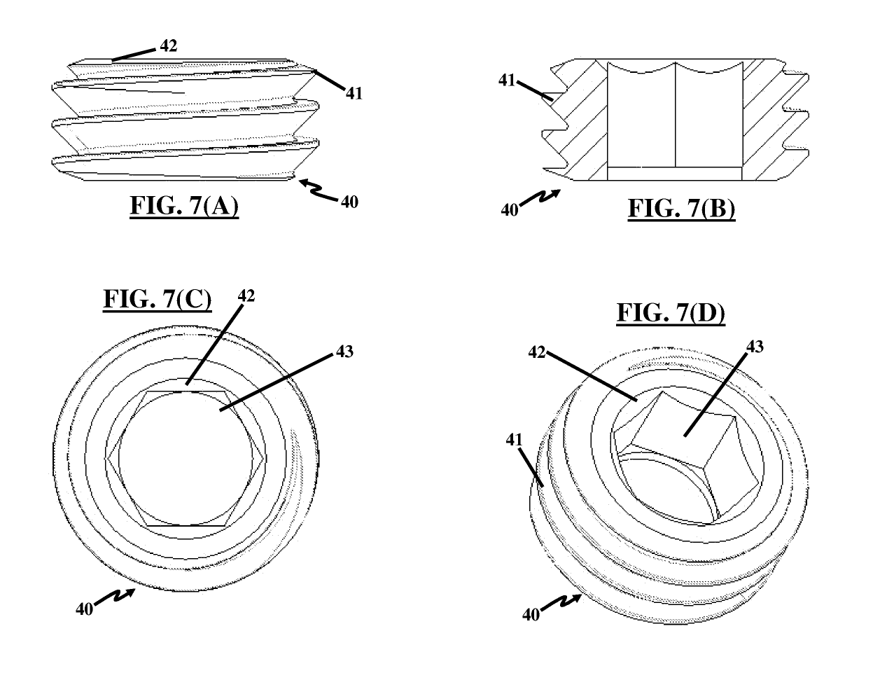

The securing member 40, which is further illustrated in FIGS. 7(A) through 7(D), with reference to FIGS. 1 through 6(D), includes a standard buttress thread 41 configured along an outer perimeter of the securing member 40. The securing member 40 helps to secure the longitudinal member 50 inside the coupling member 40. The threads 41 of the securing member 40 are configured to engage the threads 23 of the coupling member 20. Additionally, the securing member 40 aids in preventing the expansion of the coupling member 20 when torqued on the longitudinal member 50, directing the counterforce more vertically than horizontally. The top 42 of the securing member 40 has a fastening feature 43 such as a hex or square lock feature to allow high torque to be applied in locking the screw assembly 1. Furthermore, the securing member 40 may be configured with a free rotating saddle (not shown) to accommodate, via tangential contact, the longitudinal member 50 and help to further prevent notching of the titanium alloy used to construct the longitudinal member 50. Moreover, the securing member 40 may have a “timed” thread 41 that is consistently and precisely related to the securing member driving tool (not shown) to help calculate the torsional and vertical position of the securing member 40 thereby assisting the torque measurement applied to the securing member 40. In another embodiment, not shown, the securing member 40 may be configured such that the threads 41 are configured along the inner perimeter of the securing member in order to mate with threads 23 that may be configured on the outside of the upright ends 22 of the coupling member 22.

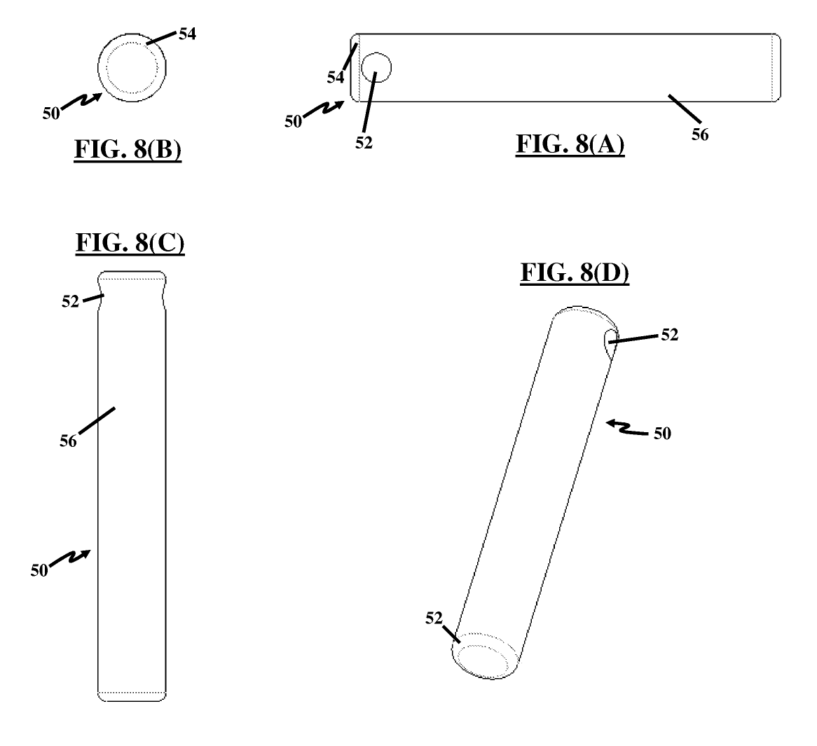

FIGS. 8(A) through 8(D), with reference to FIGS. 1 through 7(D), illustrate various views of the longitudinal member 50 of the screw assembly 1 of FIG. 1 according to an embodiment herein. As shown, longitudinal member 50 includes a body portion 56 with a plurality of hinge components (or pivot couplings) 52 cut therein. In addition, longitudinal member 50 may include a chamfered edge 54. The plurality of hinge components 52 are configured to mate with an insertion device (not shown). Such an insertion device (e.g., U.S. patent application Ser. No. 11/753,632, the complete disclosure of which, in its entirety, is herein incorporated by reference) may include nubs that can grip the hinge components 52 of the longitudinal member 50 especially when the hinge components 52 are embodied as dimples indented in the longitudinal member 50. Also, the hinge components 52 may be configured anywhere on the body portion 56 and may include any number of hinge components 52. Although longitudinal member 50 may be configured as a spinal rod, as shown in FIGS. 8(A) through 8(D); longitudinal member 50 is not limited to a spinal rod and may include any surgical implant and have any suitable configuration.

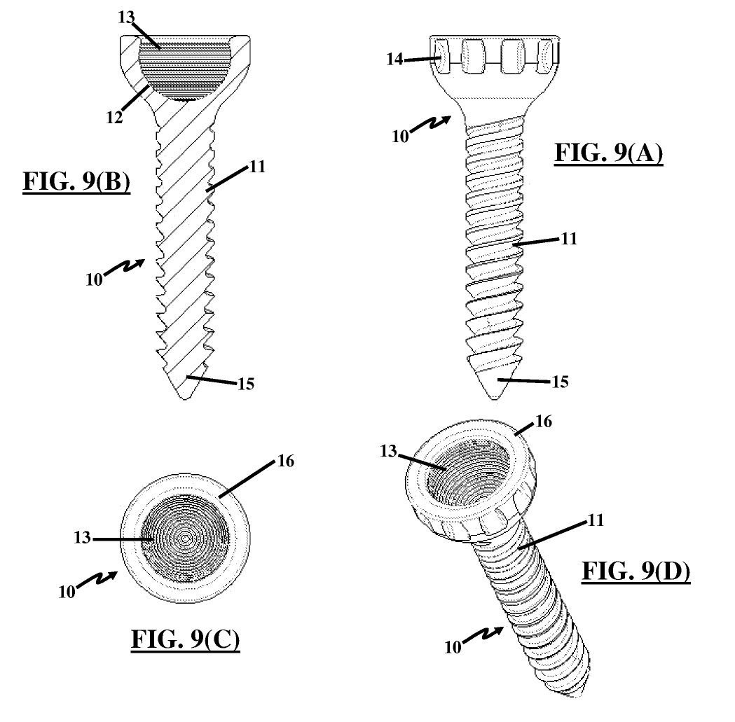

FIGS. 9(A) through 9(D), with reference to FIGS. 1 through 8(D), illustrate various views of fixator component 10 of the screw assembly 1 of FIG. 1 according to an embodiment herein. The fixator component 10 of the screw assembly 1 may have a female inner socket 12 and outer grooves 14. The fixator component 10 may have a threaded end 11, which extends from the bottom end of the female socket 12 to a pointed end 15. FIGS. 9(A) and 9(B) illustrate the side view and cross-sectional view, respectively, of the fixator component 10 having the female socket 12, the pointed end 15, the grooves 14, and the threaded end 11. The female socket 12 may have a curved surface 13. FIG. 9(C) is the top view which shows the top of the fixator component 10 having the curved surface 13 and the external annular lip 16. The fixator component 10 may include the threaded end 11 and the pointed end 15 to anchor into vertebra (not shown). The female socket 12 with the curved surface 13 is dimensioned and configured to accommodate the coupling member 20 (e.g., through the semi-bulbous male end 21 of FIGS. 4(A) through 4(D)). The grooves 14 permit the gripping of an inserter device (not shown), such as a screwdriver, to the fixator component 10. The annular lip 16 may fix a cushion joint element (not shown).

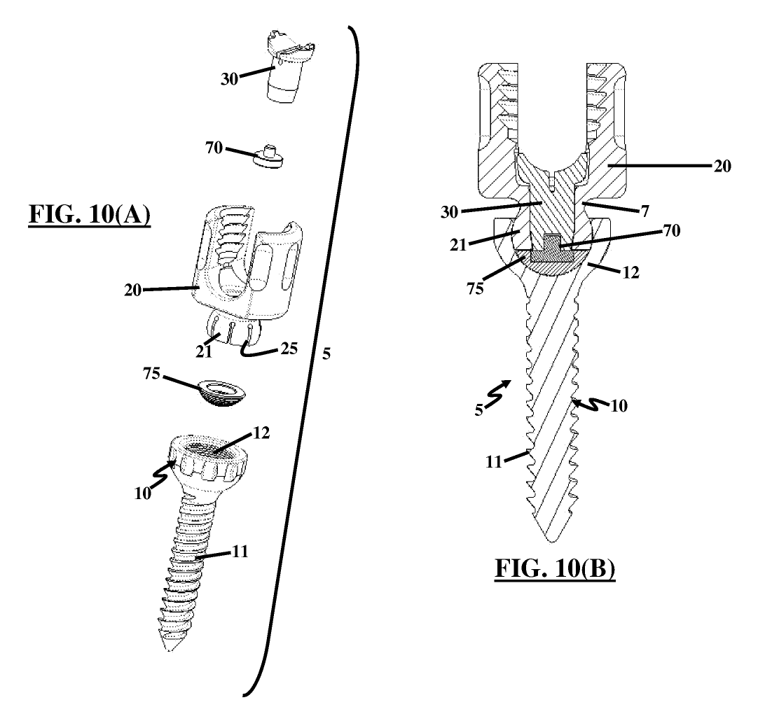

FIGS. 10(A) and 10(B), with reference to FIGS. 1 through 9(D), illustrate a pedicle screw assembly 5 according to a second embodiment herein. The screw assembly 5 comprises a fixator component (e.g., a bone screw) 10 having a threaded end 11 for engaging a bone (not shown) and a concave female socket 12 for engaging and receiving the coupling member 20. In another embodiment, the fixator component may be embodied as a hook mechanism (not shown).

FIG. 10(B) also shows that the female spherical pocket 12 of the fixator component 10 has an undercut 7 to allow the coupling member 20 to pivot freely but not to disassemble once the expanding saddle connection pin 30 is inserted. The thread 11 of the fixator component 10 may be a multiple lead thread to allow faster insertion into a bone. This thread 11 may be tapered on the minor diameter while cylindrical on the major diameter to allow a new “bite” with every turn and to accommodate more thread depth towards the bottom of the fixator component 10 for the cancellous bone.

Once the fixator component 10 is inserted into the bone (not shown), a longitudinal member 50 (shown in FIG. 1), which may be embodied as a rod, bar, etc. and securing member 40 (shown in FIG. 1) are inserted into the screw assembly 5 in order to lock the assembly 5 in place. In one embodiment herein, the coupling member 20 can accommodate 5.5 mm as well as 6.0 mm rods, which is advantageous over conventional screw assemblies that are limited to accepting only rods of a uniform dimension. FIG. 10(B) illustrates the assembled view of the screw assembly 5 in the straight monoaxial direction. In one embodiment, the threads 11 of the fixator component 10 are double lead, which provides greater surface contact with the bone, but drives at 4 mm/revolution. While not shown in FIG. 10(B), in one embodiment herein, the maximum angulation is 25 degrees/side, but the medial correction/travel of the longitudinal member 50 (shown in FIG. 1) is 3.8 mm/side, which is nearly twice of what most conventional screws offer.

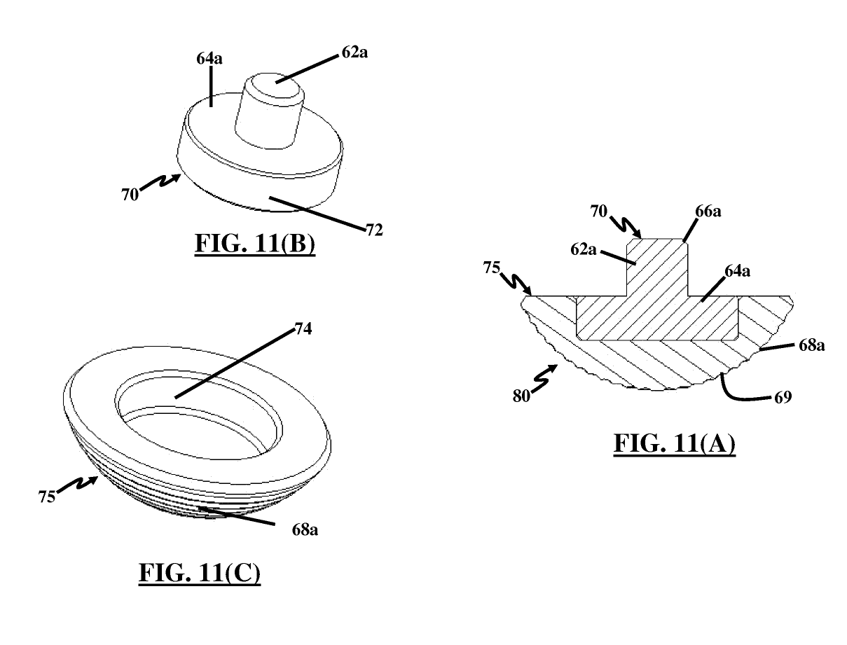

In addition, FIGS. 10(A) and 10(B) illustrate a schematic diagram of a locking process of the screw assembly 1 according to one embodiment herein. In the embodiment shown in FIGS. 10(A) and 10(B), the semi-spherical portion 21 (e.g., a polyaxial rotatable connecting end) of the coupling member 20 has slots 25 to allow the semi-spherical portion 21 to be snap-inserted into the female socket 12 of fixation component 10. In addition, the saddle connection pin 30 is placed on the coupling member 20 (e.g., in groove 26, as shown in FIGS. 4(A) through 4(D)) to prevent the coupling member 20 from disengaging the fixation component 10. As shown in FIG. 10(A), the resisting anchor 75 is placed inside the female socket 12 of the fixation component 10 and resisting cap 70 is placed at the tip of saddle pin 30. In particular, the connecting portion 62a (shown in FIG. 11(A) of the resisting cap 70 is inserted into the mating portion 38 (shown in FIGS. 4(A) through 4(D)) of the saddle connection pin 30. Consequently, saddle connection pin 30 is located between the longitudinal member 50 (shown in FIG. 1) and the combination of resisting cap 70 and resisting socket 75.

FIGS. 11(A) and 11(B), with reference to FIGS. 1 through 10(B), illustrate various views of the resisting member assembly 80 according to a second embodiment herein. As shown in the embodiment of FIGS. 11(A) through 11(C), resisting member assembly 80 can be separated into two pieces (e.g., resisting cap 70 (of FIG. 11(B)) and resisting anchor 75 (of FIG. 11(C))) to form a resisting member assembly 80 (of FIG. 11(A)). The resisting cap 70 of the resisting member assembly 80 can be attached to the saddle connection pin 30 for ease of assembly of pedicle screw assembly 5. In addition, resisting cap 70 includes a mating member 62a protruding/extending outwardly from a generally flat surface 64a to securely and evenly couple to saddle connection pin 30 (shown in FIG. 5(A) through 5(D)) via resisting member socket 38 and flat end 37. Resisting anchor 75 also includes a convexed surface 69 that in curved to create a full sphere when mated with the male semi-bulbous end 21 (shown in FIG. 10(B)). Moreover, the convexed surface 69 of resisting anchor 75 is curved to match the inner female socket 12 of the fixator component 10. Resisting anchor 75 also includes socket 74 that is configured to mate with bottom portion 72 of resisting cap 70. The mating of resisting anchor 75 and resisting cap 70 is shown in FIG. 11(A). In addition, the embodiment of resisting cap 70 includes a chamfered edge 66a of mating member 62a. As stated above, resisting anchor 75 is placed into the female socket 12 of fixator component 10 and resisting cap 75 is placed in saddle connection pin 30. To better secure resisting anchor 75 into female socket 12, one embodiment of resisting anchor 75 includes ridges 68a on the convexed surface 69. The material properties of the resisting assembly 80 are such that it prevents the deformation on the saddle connection pin 30 before the saddle connection pin 30 gives the proper bending and penetrating effects onto the coupling member 20 and fixator component 10 assembly. Examples of the types of materials used for the resisting member 60 include Zyranox™ and HIP Vitox™, both of which are available from Morgan Advanced Ceramics, United Kingdom.

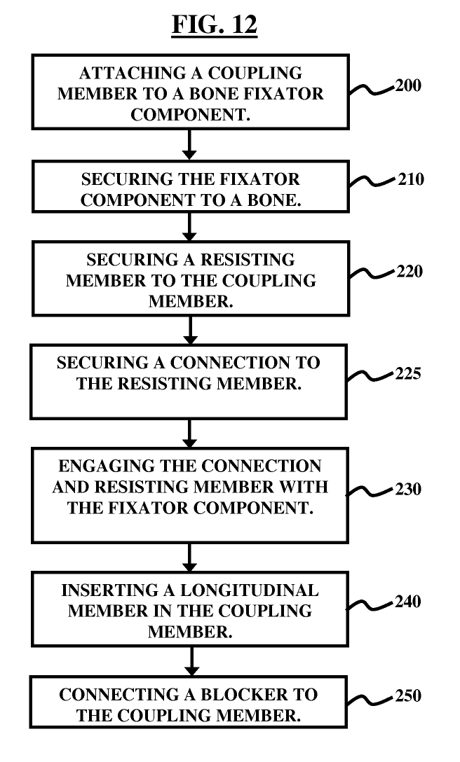

Another aspect of the embodiments herein is illustrated in the flowchart of FIG. 12, which includes descriptions which refer to components provided in FIGS. 1 through 11(C). FIG. 12 illustrates a method of assembling a pedicle screw assembly 1, 5 wherein the method comprises attaching (200) a coupling member 20 to a bone fixator component 10; securing (210) the fixator component 10 to a bone (not shown); securing (220) a resisting member 60, 80 in the coupling member 20; securing (225) a connection pin 30 to the resisting member 60, 80; engaging (230) the connection pin 30 and resisting member 60, 80 with the fixator component 10; inserting (240) a longitudinal member 50 in the coupling member 20; and connecting (250) a blocker 40 to the coupling member 20. As mentioned, the embodiments herein provide an axial movement of the coupling member 20 up to 25 degrees in any plane. Moreover, the embodiments herein allow for greater medial translation of the longitudinal member 50 (e.g., nearly 4 mm is possible, compared to the conventional devices which are generally limited to 2 mm).

Moreover, according to an aspect of the embodiments herein, the screw assembly 1, 5 can be used as a dynamic rod system to complement artificial discs. According to this embodiment, the outside of the semi-bulbous male end 21 of the coupling member 20 and the inner spherical surface of female socket 12 are coated with a wear resistant ceramic coating. In this scenario, resisting member 60, 80 and saddle connection pin 30 are not rubbing against the female socket 12 of the fixator component 10 and in fact is configured at a shorter length than some of the other embodiments. This system allows some motion instead of rigid fixation and shares the load with the artificial disc disallowing excessive forces being applied to the artificial disc and increasing its functional life. For example, this occurs as a result of the ceramic coating, which may be used in the embodiments herein. As such, the semi-bulbous male end 21 of the coupling member 20 and the female socket 12 of the fixator component 10 have lower friction and higher wear resistance characteristics, thus improving the overall characteristics of the screw assembly 1, 5.

The embodiments herein provide a pedicle screw assembly implant device 1, 5, which may be used anteriorly or posteriorly, and which is capable of being utilized in surgeries to achieve anterior lumbar interbody fusion, posterior lumbar interbody fusion, transverse lumbar interbody fusion, correct degenerative disc disease, adult and pediatric scoliosis as a fixation device, and posterior cervical fusion.

Moreover, the embodiments herein provide a polyaxial spinal screw that can become rigid similar to a monoaxial screw inter-operatively on demand. The embodiments herein also offer the surgeon more lateral range of motion than conventional products by utilizing the space under the screw head to provide a bigger arc of rotation. Moreover, the saddle connection pin 30 component offers the flexibility to use a diametrical range of spinal longitudinal members 50 instead of a fixed size longitudinal member.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. An assembly comprising:
a coupling member comprising a semi-bulbous end;
a fixator component that receives said semi-bulbous end of said coupling member;
a resisting member mounted in said coupling member, said resisting member comprising a mating member;
a connection pin comprising a resisting member socket operatively connected to said mating member of said resisting member; and
a blocker that engages said coupling member.

2. The assembly of claim 1, wherein said coupling member comprises:
a slot that receives a longitudinal member; and
a bottom portion operatively connected to said semi-bulbous end.

3. The assembly of claim 1, wherein said fixator component comprises a concave socket that receives said semi-bulbous end of said coupling member.

4. The assembly of claim 1, wherein said fixator component comprises an anchor end opposite said concave socket, and wherein said anchor end attaches to a bone.

5. The assembly of claim 2, wherein said connection pin engages said fixator component and a bottom portion of said longitudinal member.

6. The assembly of claim 2, wherein said blocker secures a top portion of said longitudinal member.

7. The assembly of claim 1, wherein said semi-bulbous end comprises a coupling member flat end, and wherein said semi-bulbous end sits flush with said resisting member.

8. The assembly of claim 1, wherein said connection pin comprises a connection pin flat end, and wherein said connection pin flat end sits flush with said resisting member.

9. The assembly of claim 1, wherein said resisting member comprises a mechanically harder material than said connection pin.

10. The assembly of claim 9, wherein said coupling member and said fixator component comprise a first material, and wherein said resisting member comprises a material having a higher material hardness and compressive yield strength than said first material.

11. The assembly of claim 1, wherein said resisting member comprises a plurality of separately configured parts matingly attached together.

12. The assembly of claim 2, wherein said coupling member comprises a plurality of opposed upright ends separated by said slot.

13. The assembly of claim 12, wherein each of said opposed upright ends comprise an inner wall and an outer wall, wherein any of said inner wall and said out wall comprises wall threads, and wherein said outer wall comprises grooves.

14. The assembly of claim 13, wherein said blocker comprises blocker threads dimensioned and configured to mate with said wall threads.

15. The assembly of claim 1, wherein said semi-bulbous end of said coupling member comprises a plurality of slots terminating at an opening at a tip of said semi-bulbous end.

16. The assembly of claim 1, wherein said semi-bulbous end of said coupling member comprises a gap that receives said connection pin.

17. A pedicle fixation assembly comprising:
a screw head comprising a polyaxial rotatable connecting end comprising a curved outer surface that forms a semi-bulbous body, wherein said semi-bulbous body comprises a substantially first flat surface;
a resisting member comprising:
a substantially second flat surface sitting flush with said first flat surface; and
a convexed surface curved to match a contour of said curved outer surface of said polyaxial rotatable connecting end of said screw head;
a bone fixator component comprising a female concave semi-spherical socket for receiving said screw head;
a pin for engaging said screw head, said resisting member, and said bone fixator component; and
a blocker for engaging said screw head and for securing said longitudinal member.

18. The assembly of claim 17, wherein said resisting member comprises:
a resisting cap; and
a resisting anchor comprising a socket for receiving said resisting cap.

19. A method of assembling a pedicle fixation assembly, said method comprising:
attaching a coupling member to a bone fixator component;
securing said bone fixator component to a bone;
securing a resisting member in said coupling member;
securing a connection pin to said coupling member;
engaging said connection pin and said resisting member with said fixator component;
inserting a longitudinal member in said coupling member; and
connecting a blocker to said coupling member.

20. The method of claim 18, wherein said coupling member comprises a male semi-spherical end and said fixator component comprises a female concave semi-spherical socket for receiving said coupling member.
